(19) United States
(12) Patent Application Publication
(10) Pub. No.: US 2017/0367569 A1
(43) Pub. Date: Dec. 28, 2017

(54) ILLUMINATION APPARATUS, ENDOSCOPE AND ENDOSCOPE SYSTEM

(71) Applicant: OLYMPUS CORPORATION, Tokyo (JP)

(72) Inventors: Satoshi OHARA, Hachioji-shi (JP); Takeshi ITO, Hino-shi (JP); Bakusui DAIDOJI, Hachioji-shi (JP)

(73) Assignee: OLYMPUS CORPORATION, Tokyo (JP)

(21) Appl. No.: 15/677,602

(22) Filed: Aug. 15, 2017

Related U.S. Application Data
(63) Continuation of application No. PCT/JP2015/062425, filed on Apr. 23, 2015.

(51) Int. Cl. A61B 1/07; A61B 1/018; A61B 1/00; A61B 1/12; A61B 1/06

(52) U.S. Cl. CPC A61B 1/07; A61B 1/128; A61B 1/0669; A61B 1/00096; A61B 1/018; A61B 1/0623

(57) ABSTRACT

An illumination apparatus includes a light converter disposed on a distal end surface of a light guide, the light converter being configured to emit illumination light, which is generated by converting optical characteristics of primary light that is guided by the light guide, in a forward direction which is on the light converter side of the distal end surface, and in a backward direction which is on the light guide side of the distal end surface. The illumination apparatus further includes a light collector configured to collect backward illumination light into the light guide such that the backward illumination light is guided backward by the light guide, and a heat exhauster configured to convert the backward illumination light, which is guided by the light guide, to heat, and to exhaust the heat.

### CROSS-REFERENCE TO RELATED APPLICATIONS

This application is a Continuation Application of PCT Application No. PCT/JP2015/062425, filed Apr. 23, 2015, the entire contents of all of which are incorporated herein by reference.

### BACKGROUND OF THE INVENTION

### 1. Field of the Invention

The present invention relates to an illumination apparatus, an endoscope and an endoscope system.

### 2. Description of the Related Art

For example, Jpn. Pat. Appin. KOKAI Publication No. 2011-248022 discloses an illumination apparatus which includes a single optical fiber. The illumination apparatus includes an ellipsoidal diffusion body which serves as a light converter disposed on a distal end surface of the optical fiber, in order to convert a laser beam, which is primary light guided by the optical fiber, to illumination light which is irradiated in a wide range.

### BRIEF SUMMARY OF THE INVENTION

According to one embodiment of the invention, an illumination apparatus includes a light source module configured to emit primary light; a light guide configured to guide the primary light emitted from the light source module;

a light converter disposed on a distal end surface of the light guide, the light converter being configured to emit illumination light, which is generated by converting optical characteristics of the primary light that is guided by the light guide, in a forward direction which is on the light converter side of the distal end surface, and in a backward direction which is on the light guide side of the distal end surface; a light collector configured to collect backward illumination light, which is the illumination light emitted backward from the light converter, into the light guide, such that the backward illumination light is guided backward by the light guide; and a heat exhauster configured to convert the backward illumination light, which is guided by the light guide, to heat, and to exhaust the heat.

Additional objects and advantages of the invention will be set forth in the description which follows, and in part will be obvious from the description, or may be learned by practice of the invention. The objects and advantages of the invention may be realized and obtained by means of the instrumentalities and combinations particularly pointed out hereinafter.

### BRIEF DESCRIPTION OF THE SEVERAL VIEWS OF THE DRAWINGS

The accompanying drawings, which are incorporated in and constitute a part of the specification, illustrate embodiments of the invention, and together with the general description given above and the detailed description of the embodiments given below, serve to explain the principles of the invention.

FIG. 1A is a schematic view of an illumination apparatus according to a first embodiment of the present invention.

FIG. 1B is a view illustrating the configuration of a distal end portion of a light guide and a light converter.

FIG. 1C is a view illustrating the configuration of a light source portion and a heat exhauster.

FIG. 2A is a view for explaining Mie scattering.

FIG. 2B is a view for explaining Rayleigh scattering.

FIG. 3A is a view illustrating the configuration of a distal end surface of a light guide according to Modification 1 of the first embodiment.

FIG. 3B is a view illustrating the configuration of a distal end surface of a light guide according to Modification 2 of the first embodiment.

FIG. 4A is a view illustrating the configuration of a distal end portion of a light guide and a light converter according to a second embodiment of the present invention.

FIG. 4B is a view illustrating the configuration of a light source portion and a heat exhauster according to the second embodiment.

FIG. 5A is a view illustrating the configuration of a distal end portion of a light guide and a light converter according to a third embodiment of the present invention.

FIG. 5B is a view illustrating a modification of the configuration of the distal end portion of the light guide and the light converter.

FIG. 5C is a side view of the configuration illustrated in FIG. 5B.

FIG. 5D is a view illustrating the configuration of a light source portion and a heat exhauster according to the third embodiment.

FIG. 6A is a view illustrating a fourth embodiment of the present invention, FIG. 6A being a schematic perspective view of an endoscope system including the illumination apparatus according to the first embodiment.

FIG. 6B is a view illustrating the configuration of the endoscope system illustrated in FIG. 6A.

FIG. 7A is a view illustrating Modification 1 of the fourth embodiment of the invention, FIG. 7A being a schematic perspective view of an endoscope system including an endoscope in which the illumination apparatus according to the first embodiment is mounted.

FIG. 7B is a view illustrating the configuration of the endoscope system illustrated in FIG. 7A.

FIG. 8A is a view illustrating Modification 2 of the fourth embodiment of the invention, FIG. 8A being a schematic perspective view of an endoscope system including an endoscope in which the illumination apparatus according to the first embodiment is mounted.

FIG. 8B is a view illustrating the configuration of the endoscope system illustrated in FIG. 8A.

### DETAILED DESCRIPTION OF THE INVENTION

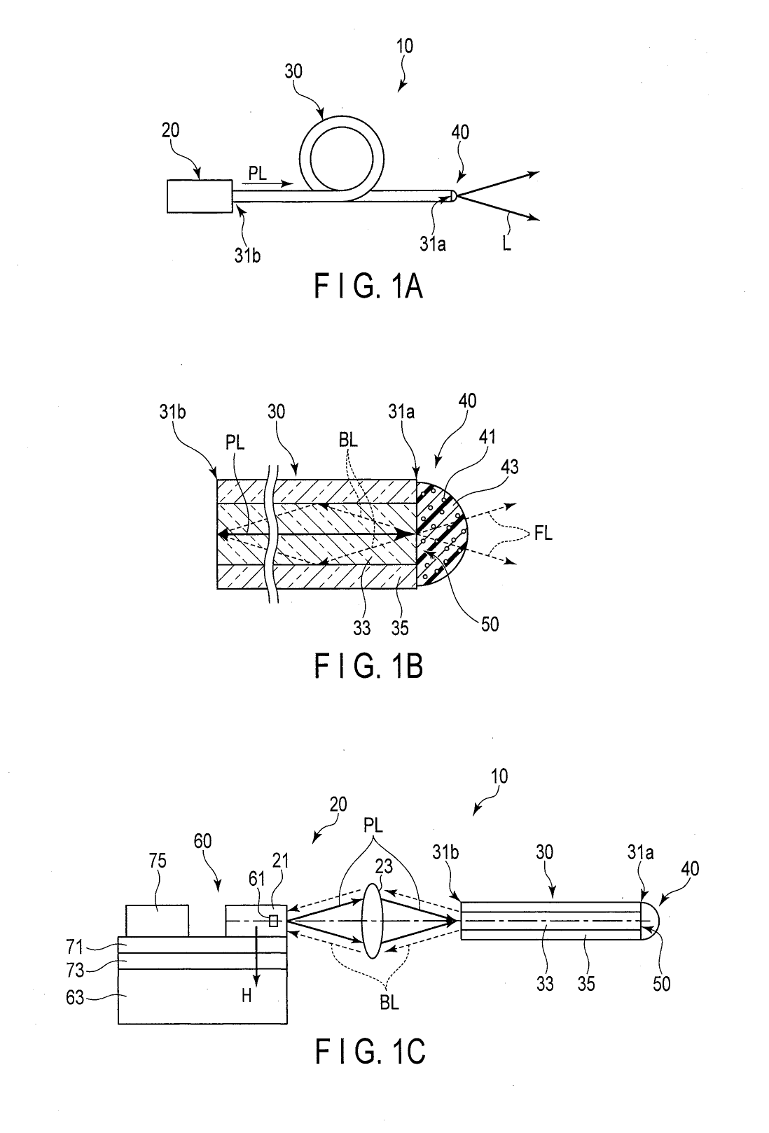

Hereinafter, embodiments of the present invention will be described in detail with reference to the accompanying drawings. Incidentally, in some of the drawings, depiction of some members is omitted for the purpose of clearer illustration, such as omission of depiction of diffusion particles 41 in FIG. 1A.

### First Embodiment

[Configuration]

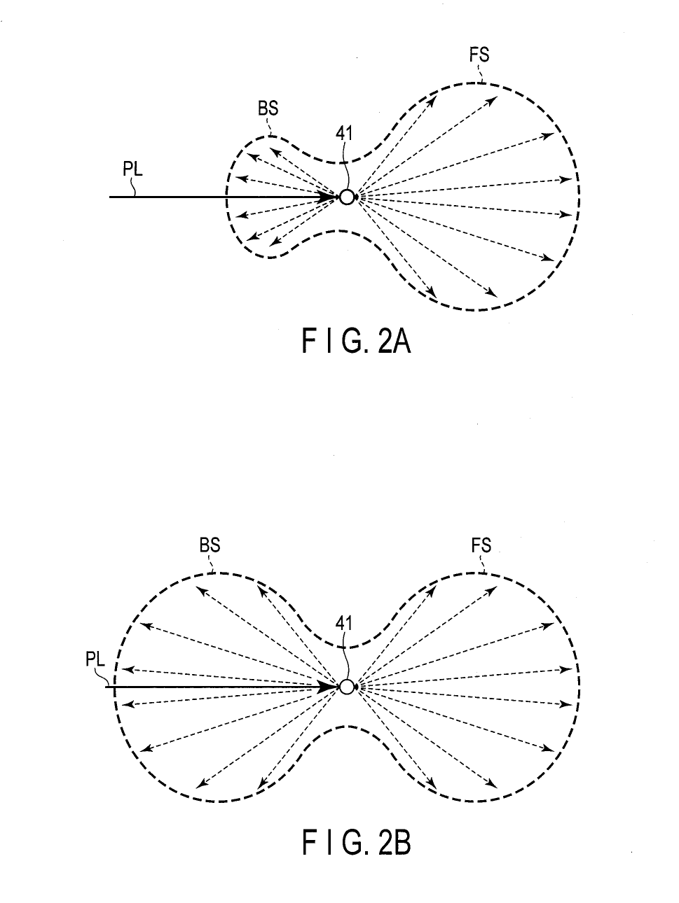

A first embodiment will be described with reference to FIG. 1A, FIG. 1B, FIG. 1C, FIG. 2A, and FIG. 2B.

[Configuration 1 of Illumination Apparatus 10]

As illustrated in FIG. 1A, FIG. 1B and FIG. 1C, an illumination apparatus 10 includes a light source module 20 which emits primary light PL such as a laser beam; a light guide (light guide member) 30 which guides the primary light PL which is emitted from the light source module 20; and a light converter (light conversion portion) 40 which is disposed on a distal end surface 31a of the light guide 30.

[Light Source Module 20]

As illustrated in FIG. 1C, the light source module 20 includes a light source 21 which emits the primary light PL; and a light focusing portion 23 which focuses the primary light PL, which is emitted from the light source 21, onto the light guide 30.

As illustrated in FIG. 1C, the light focusing portion 23 includes a light focusing lens which focuses the primary light PL onto a proximal end surface 31b of the light guide 30. The proximal end surface 31b is a surface on a side opposite to the distal end surface 31a.

[Light Guide 30]

The light guide 30 as illustrated in FIG. 1A, FIG. 1B and FIG. 1C includes an optical fiber. The light guide 30 is preferable that the light guide 30 is, for example, a multi-mode optical fiber which guides a plurality of modes of the primary light PL and backward illumination light BL (to be described later). The optical fiber may be a single-mode optical fiber. The material of the light guide 30 is, for instance, silica glass, plastic, or resin. The light guide 30 is a bendable rod-like member. The distal end surface 31a is perpendicular to a center axis of the light guide 30, and a side surface of the light guide 30 is parallel to the center axis of the light guide 30. The distal end surface 31a may be formed by cutting the light guide 30 by a general cleaver, or may be formed by polishing the light guide 30 after cleaving. The distal end surface 31a is smooth. It is preferable that the NA of the light guide 30 is high. Specifically, the NA is 0.22 or more.

As illustrated in FIG. 1B and FIG. 1C, the light guide 30 includes a core 33 which guides the primary light PL and the backward illumination light BL, and a cladding 35 which is provided on an outer periphery of the core 33 and has a refractive index that is lower than the refractive index of the core 33. The cladding 35 has a function of confining the primary light PL in the core 33. A distal end surface of the core 33, which is included in the distal end surface 31a, is a planar surface. The refractive index of the core 33 is substantially equal to or higher than the refractive index of a contact part of the light converter 40, the contact part being in contact with the distal end surface of the core 33.

The distal end surface 31a includes the distal end surface of the core 33, and a distal end surface of the cladding 35, which is flush with the distal end surface of the core 33. The distal end surface 31a, the distal end surface of the core 33, and the distal end surface of the cladding 35 are planar.

[Light Converter 40]

The light converter 40 of the present embodiment, as illustrated in FIG. 1A, FIG. 1B and FIG. 1C, emits illumination light L, which is generated by converting optical characteristics of the primary light PL that is guided by the light guide 30, in a forward direction which is on the light converter 40 side of the distal end surface 31a, and in a backward direction which is on the light guide 30 side of the distal end surface 31a. The light converter 40 functions, for example, as a light distribution converter (light distribution conversion portion) which converts a light distribution of the primary light PL which is emitted from the light guide 30. Thus, the light converter 40 includes one or more diffusion particles 41 which diffuse the primary light PL that is emitted from the core 33, and an enclosing member 43 which encloses the diffusion particles 41 together in the state in which the diffusion particles 41 are dispersed. The diffusion particles 41 are dispersed in the inside of the enclosing member 43, and are sealed by the enclosing member 43. The light converter 40, which includes the distal end surface 31a, functions as a diffusion member.

The diffusion particles 41 are fine particles formed of a metal or a metal compound. Such diffusion particles 41 are, for instance, alumina or titanium oxide. The grain size of the diffusion particles 41 is several μm. Incidentally, fluorescent particles may be used in place of the diffusion particles 41. The fluorescent particles absorb the primary light PL, and generate fluorescence of a wavelength which is different from the wavelength of the primary light PL. However, since the generated fluorescence travels also in directions other than the forward direction, it can be said that the fluorescent particles are diffusion particles in a broad sense.

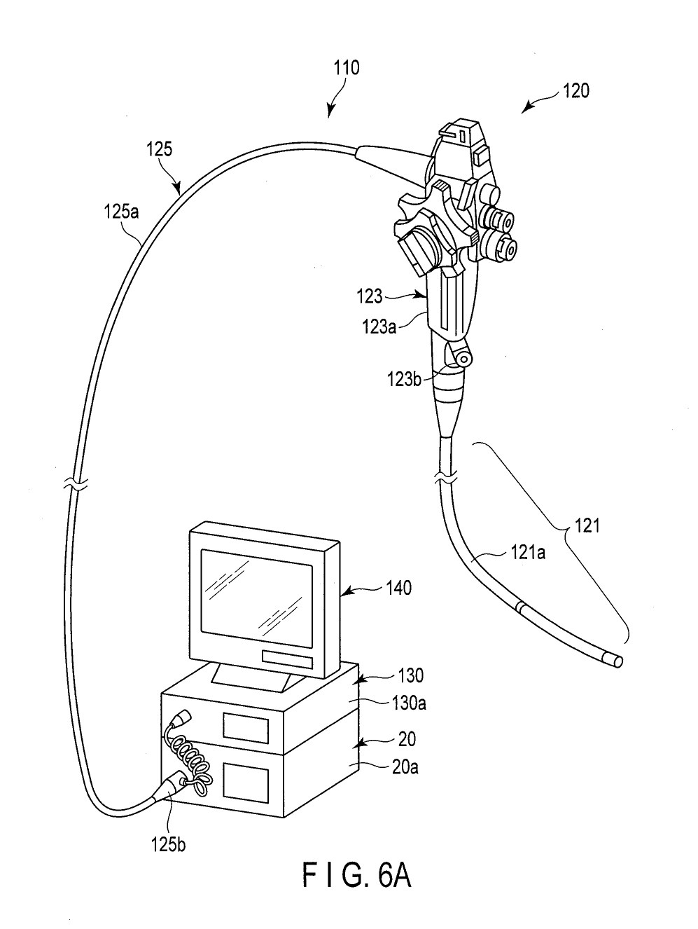

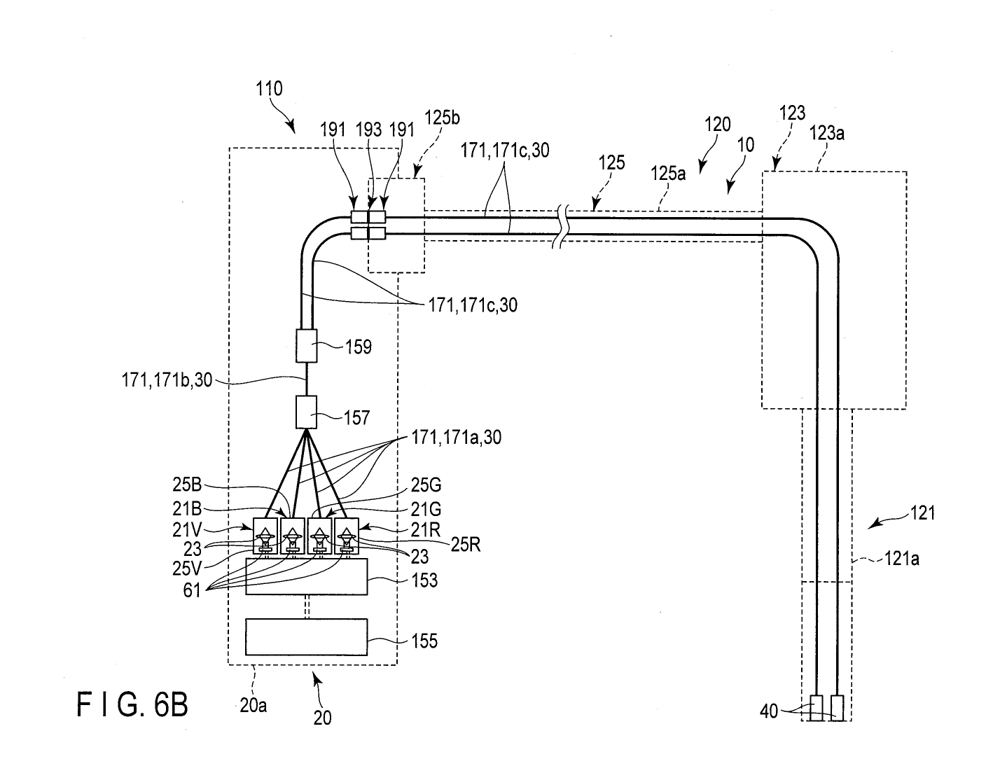

The absorptance of the diffusion particles 41 with respect to the primary light PL is preferably, for example, 20% or less, and is more preferably 10% or less. Thereby, for example, when the light converter 40 functions as the light distribution converter, the diffusion particles 41 can absorb a small light amount, and can efficiently convert the primary light PL to illumination light L. Since the amount of absorbed primary light PL decreases, heat generation can be reduced. The distal end portion of the light guide 30 and the light converter 40, which are a distal end portion of the illumination apparatus 10, are built in the distal end portion of an insertion module 121 which is provided in an endoscope 120 (see FIG. 6A and FIG. 6B). If the temperature of the light converter 40 rises, the temperature of the distal end portion of the insertion module 121 rises due to heat. In some cases, the heat of the distal end portion affects a conduit (conduit portion) through which the insertion module 121 is inserted. However, in the present embodiment, since the rise in temperature of the light converter 40 is suppressed, such concern can be reduced. For example, the conduit is a lumen of a patient.

The refractive index of the diffusion particles 41 is different from the refractive index of the enclosing member 43. For example, it is preferable that the refractive index of the diffusion particles 41 is higher than the refractive index of the enclosing member 43, and is 1.5 or more. Thereby, the diffusion particles 41 can enhance the diffusivity of the primary light PL.

The light distribution angle of the light converter 40 is controlled by, for example, the density of diffusion particles 41 relative to the enclosing member 43, the thickness of the light converter 40, etc.

The enclosing member 43 is formed of a member which transmits the primary light PL. Such enclosing member 43 is, for example, a transparent silicone resin or a transparent epoxy resin. The enclosing member 43 has a high transmittance with respect to the primary light PL. The enclosing member 43 seals the diffusion particles 41.

As illustrated in FIG. 1B, the light converter 40 is formed, for example, in a dome shape. In a concrete formation method, the enclosing member 43 prior to curing, which encloses the diffusion particles 41, is coated on the distal end surface 31a. The enclosing member 43 is formed in a dome shape due to a surface tension of the enclosing member 43. By the amount of coating being controlled, the curvature of the dome is controlled. By the enclosing member 43 being cured, the light converter 40 is formed. It is preferable that, in the cross section in the optical axis direction of the light guide 30, the central angle of the outer arc of the light converter 40 having the dome shape is 180 degrees or less. Thereby, the light converter 40 is prevented from flowing out to the side surface of the light guide 30 from the distal end surface 31a. The optical axis means the center axis of the illumination light L which is emitted in the forward direction from the distal end surface 31a.

[Diffusion Phenomenon]

Here, referring to FIG. 2A and FIG. 2B, a diffusion phenomenon will be described. In order to make the description simpler, the behavior of the primary light PL at a time when the primary light PL is incident on one diffusion particle 41 will be illustrated.

Diffusion phenomena are generally classified into Mie scattering illustrated in FIG. 2A, and Rayleigh scattering illustrated in FIG. 2B.

The Mie scattering illustrated in FIG. 2A occurs when the diameter of the diffusion particle 41 is substantially equal to the wavelength of the primary light PL. In the Mie scattering, a forward scattering component FS, which is indicative of a component of forward scattering of the primary light PL, is large, and a backward scattering component BS, which is indicative of a component of backward scattering of the primary light PL, is small.

The Rayleigh scattering illustrated in FIG. 2B occurs when the diameter of the diffusion particle 41 is about 1/10 of the wavelength of the primary light PL. In the Rayleigh scattering, the forward scattering component FS is substantially equal to the backward scattering component BS.

If consideration is given to the luminance of the forward illumination light FL which is emitted in the forward direction from the distal end surface 31a, it is preferable to utilize the Mie scattering in which the forward scattering component FS is greater than the backward scattering component BS. On the other hand, when primary light PL of multiple colors is scattered, the wavelength dependency of scattering needs to be considered. It is generally thought that the wavelength dependency of Mie scattering is greater than the wavelength dependency of Rayleigh scattering. In order to eliminate non-uniformity in color of the forward illumination light FL, the Rayleigh scattering is preferable.

In this manner, the setting of the diameter of the diffusion particle 41 is selected in accordance with the purpose of use. In the present embodiment, it is assumed that the illumination apparatus 10 uses Mie scattering. Thus, the diameter of the diffusion particle 41 is, for example, about 1/10 or more of the wavelength of the primary light PL. Specifically, when the wavelength of the primary light PL, which is used as the illumination light L, is, for example, about 400 nm to about 800 nm, the diameter of the diffusion particle 41 is 40 nm or more.

In the description thus far, the diffusion phenomenon of one diffusion particle 41 has been described. In the light converter 40 of the present embodiment, many diffusion particles 41 are enclosed in the enclosing member 43. The diffusion phenomenon of such light converter 40 is substantially the same as the diffusion phenomenon of one diffusion particle 41.

[Configuration 2 of Illumination Apparatus 10]

As illustrated in FIG. 1B, the illumination apparatus 10 further includes a light collector (light collect portion) 50 which collects backward illumination light BL into the light guide 30, such that the illumination light emitted backward (hereinafter referred to as backward illumination light BL) from the light converter 40 is guided backward by the light guide 30. The light collector 50 collects the backward illumination light BL into the light guide 30 which is provided behind the light collection portion 50. The light collector 50 includes the distal end surface of the core 33 in the distal end surface 31a and the light converter 40.

The light guide 30 has a reception angle which is defined by the NA. The backward illumination light BL, which is made incident on the core 33 by the light collector 50 at an angle of not greater than the reception angle, is guided by the light guide 30 toward the light source 21 while being repeatedly reflected in the inside of the light guide 30. Specifically, the backward illumination light BL is guided in a direction reverse to the direction of travel of the primary light PL, and reversely travels in the light guide 30 in the direction reverse to the direction of the travel of the primary light PL.

In the meantime, the backward illumination light BL, which is made incident on the core 33 at an angle of greater than the reception angle, is unable to reflect at the interface between the core 33 and cladding 35, and leaks out from the light guide 30 to the outside. Thus, in order to guide the backward illumination light BL up to the light source 21, it is preferable that the NA of the light guide 30 is as large as possible. Specifically, if the NA of the light guide 30 is greater than the incidence angle of the backward illumination light EL on the core 33, the entire backward illumination light BL can be received in the light guide 30.

In order to make a greater amount of backward illumination light EL incident on the core 33, it is preferable that the cross-sectional area of the core 33 is large and the cross-sectional area of the cladding 35 is small. For example, the diameter of the cladding 35 is not greater than 1.1 times the diameter of the core 33.

It is preferable that the refractive index of the core 33 is equal to or greater than the refractive index of the enclosing member 43. The material of the core 33 is, for example, silica glass, and the refractive index of the core 33 is, for example, 1.46. The material of the enclosing member 43 is, for example, silicone resin, and the refractive index of the enclosing member 43 is, for example, 1.5.

For example, the diameter of the core 33 is 100 μm, the diameter of the cladding 35 is 110 μm, and the NA is 0.22 or more. The optical fiber is a multi-mode optical fiber which guides a plurality of modes of the primary light PL and backward illumination light BL. The optical fiber has such an NA that the optical fiber receives 20% or more of the backward illumination light BL which is emitted backward by the light converter 40.

[Configuration 3 of Illumination Apparatus 10]

As illustrated in FIG. 1C, the illumination apparatus 10 further includes a heat exhauster (heat exhaust portion) 60. which converts the backward illumination light BL, that is guided by the light guide 30, to heat H, and which exhausts the heat H. For example, when the light converter 40 is built in the distal end portion (see FIG. 6A and FIG. 6B) of the insertion module 121, as described above, the heat exhauster 60 is provided in the light source 21 of the light source module 20 to which a universal cord 125 of the endoscope 120 is connected. In this manner, the heat exhauster 60 is provided apart from the light converter 40 and the position of diffusion. The heat exhauster 60 is provided on a side opposite to the light converter 40 via the light guide 30.

As illustrated in FIG. 1C, the heat exhauster 60 includes a heat converter (heat conversion portion) 61 which absorbs the backward illumination light BL and converts the absorbed backward illumination light BL to heat H; and a heat radiator (heat radiation portion) 63 which radiates the heat H.

As illustrated in FIG. 1C, in the light source 21 on which the backward illumination light BL, after guided by the light guide 30, is irradiated by the light focusing portion 23, the heat converter 61 is a light emission element of the light source 21, which is included in the light source module 20 and emits the primary light PL. The heat converter 61 is thermally connected to the heat radiator 63 via a base plate 71 and a Peltier element 73. The heat H, which is generated from the light source 21 in accordance with the emission of the primary light PL, and the heat H, which is generated from the light source 21 by the irradiation of the backward illumination light BL, are transferred to the heat radiator 63 via the base plate 71 and Peltier element 73.

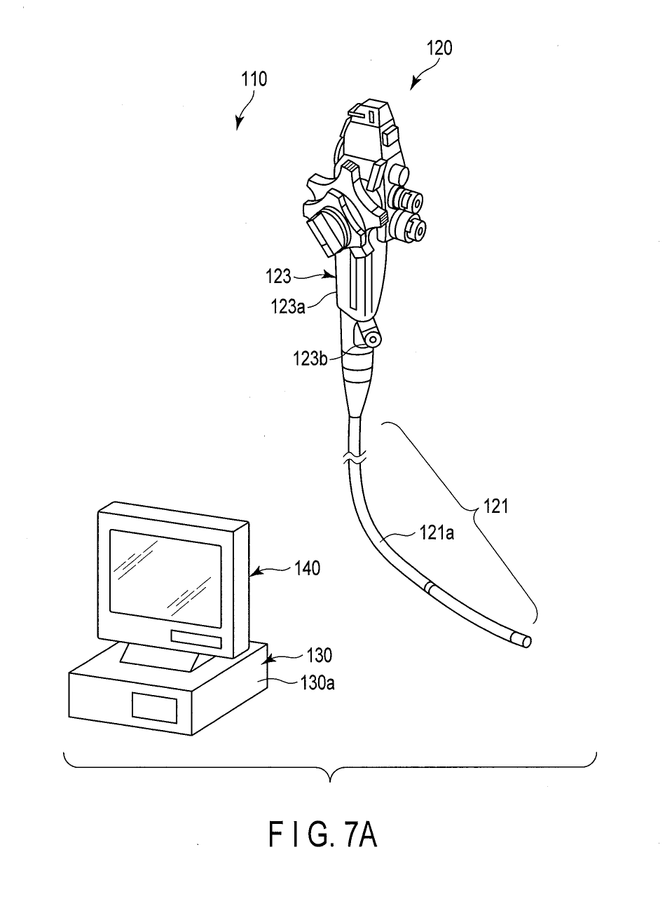

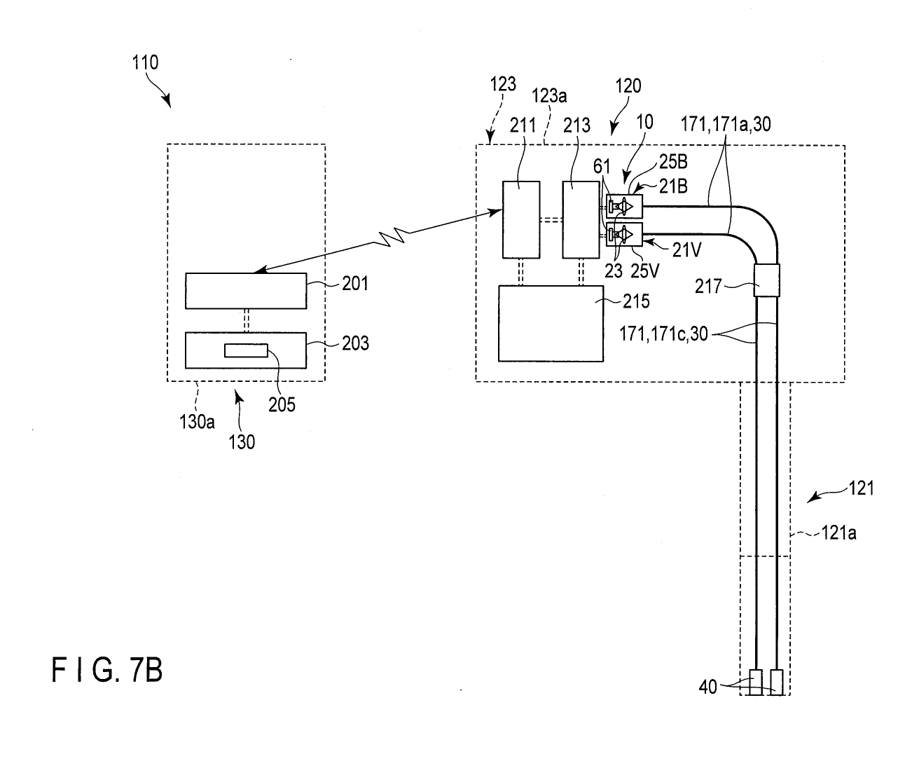

The heat radiator 63 radiates heat to the outside. Incidentally, as illustrated in FIG. 7B, when light sources 21V and 21B (to be described later) are provided in the inside of the endoscope 120, the heat exhauster 60, the depiction of which is omitted in FIG. 7B, is also provided in the inside of the endoscope 120. In this case, the “outside” means an atmosphere within the endoscope 120.

The temperature of the heat conversion member 61 is measured by a temperature measuring sensor (temperature measuring portion) 75 which is mounted on the base plate 71. The temperature measuring sensor 75 includes, for example, a thermistor. If the heat conversion member 61 is irradiated with the backward illumination light BL, there is concern that the operation of the heat converter 61 becomes unstable. As a result, there is concern that the emission of the primary light PL becomes unstable. By the temperature measuring sensor 75 measuring the temperature of the heat converter 61, heat transfer to the Peltier element 73 is properly performed for the heat converter 61, and the operation of the heat converter 61 is stabilized.

[Function]

As illustrated in FIG. 1C, the primary light PL is emitted from the light source 21 and is focused on the light guide 30 by the light focusing portion 23. The primary light PL is guided by the light guide 30, and travels to the light converter 40. As illustrated in FIG. 1B, the light converter 40 diffuses the primary light PL, and the forward illumination light FL and backward illumination light BL are generated. The forward illumination light FL irradiates a to-be-illuminated portion.

As illustrated in FIG. 1B, the backward illumination light BL is collected into the core 33 by the light collector 50. Thus, when the primary light PL is guided by the light guide 30 and is then diffused, the backward illumination light BL is exactly made incident on the light guide 30. Since the backward illumination light BL is neither irradiated on, nor absorbed by, other members near the light converter 40, a temperature rise of the distal end portion of the insertion module 121, which includes these other members, can be suppressed. Accordingly, when the insertion module 121 is inserted in, for example, a conduit, even if the distal end portion comes indirect contact with the conduit, there is no concern that the conduit is damaged by heat. In this manner, in the present embodiment, since the temperature rise of other members near the light converter 40 is suppressed, the influence on the conduit by the heat can be reduced.

As illustrated in FIG. 1C, the backward illumination light BL is guided by the light guide 30, and irradiates the light source 21 via the light focusing portion 23. At this time, the backward illumination light BL is guided in the direction reverse to the direction of the travel of the primary light PL, reversely travels in the light guide 30 in the direction reverse to the direction of the travel of the primary light PL, and returns to the light source 21. The light emission element of the light source 21, which is the heat converter 61, absorbs the backward illumination light BL and converts the absorbed backward illumination light BL to heat H. This heat H is radiated to the outside by the heat radiator 63 via the base plate 71 and Peltier element 73. This “outside” means, for example, an outside environment of the endoscope 120, or an atmosphere within the endoscope 120.

The heat converter 61 and heat radiator 63 convert light to heat H at a location apart from the light converter 40, and radiates the heat H at a location apart from the light converter 40. Thus, in the present embodiment, the heat generation of the distal end portion of the insertion module 121 at a time of illumination, in which the light converter 40 is provided, can be suppressed to a minimum.

[Advantageous Effects]

As described above, in the present embodiment, when the light is guided by the light guide 30 and is then diffused, the backward illumination light BL is exactly made incident on the light guide 30, without the backward illumination light BL being absorbed by other members near the light converter 40. In addition, the light can be converted to the heat H at a location apart from the light converter 40, by the light guide 30 and heat exhauster 60. Thereby, the heat generation of the distal end portion of the insertion module 121 at a time of illumination, in which the light converter 40 is provided, can be suppressed to a minimum.

The light guide 30 guides the primary light PL and backward illumination light BL. Thus, compared to a case in which a light guide 30 for primary light PL and a light guide 30 for backward illumination light BL are provided separately from each other, the number of structural parts can be reduced and the configuration can be simplified. When the illumination apparatus 10 is mounted in the endoscope 120, a contribution can be made to the reduction in diameter of the insertion module 121.

The heat exhauster 60 converts the backward illumination light BL, which is guided by the light guide 30, to heat H, and exhausts the heat H. This heat exhauster 60 is provided on the side opposite to the light converter 40 via the light guide 30. Hence, the light can be converted to the heat H at a location apart from the light converter 40, and the heat H can be radiated at a location apart from the light converter 40.

The heat converter 61 is a light emission element of the light source 21. Thus, compared to a case in which a heat conversion member 61, which is different from the light emission element, is provided as one member, the number of structural parts can be reduced and the configuration can be simplified.

In the meantime, when the light guide 30 is provided in the inside of the insertion module 121 which has a diameter of, for example, ten-odd mm, the light guide 30 guides 5% or more of the backward illumination light BL which is emitted backward by the light converter 40. In addition, the heat exhauster 60 converts 5% or more of the backward illumination light BL, which is emitted backward by the light converter 40, to the heat H. Thereby, the temperature of the distal end portion of the insertion module 121 can be prevented from rising up to a dangerous range.

When the light guide 30 is provided in the inside of the insertion module 121 which has a diameter of, for example, 5 mm to 10 mm, the light guide 30 guides 10% or more of the backward illumination light BL which is emitted backward by the light converter 40. In addition, the heat exhauster 60 converts 10% or more of the backward illumination light BL, which is emitted backward by the light converter 40, to the heat H. Thereby, the temperature of the distal end portion of the insertion module 121 can be prevented from rising up to a dangerous range.

When the light guide 30 is provided in the inside of the insertion module 121 which has a diameter of, for example, 5 mm or less, the light guide 30 guides 20% or more of the backward illumination light BL which is emitted backward by the light converter 40. In addition, the heat exhauster 60 converts 20% or more of the backward illumination light BL, which is emitted backward by the light converter 40, to the heat H. Thereby, the temperature of the distal end portion of the insertion module 121 can be prevented from rising up to a dangerous range.

Incidentally, the distal end surface 31a of the light guide 30 does not need to be limited to a planar surface. Hereinafter, configurations of the distal end surface 31a will be described as Modifications 1 and 2.

[Modification 1]

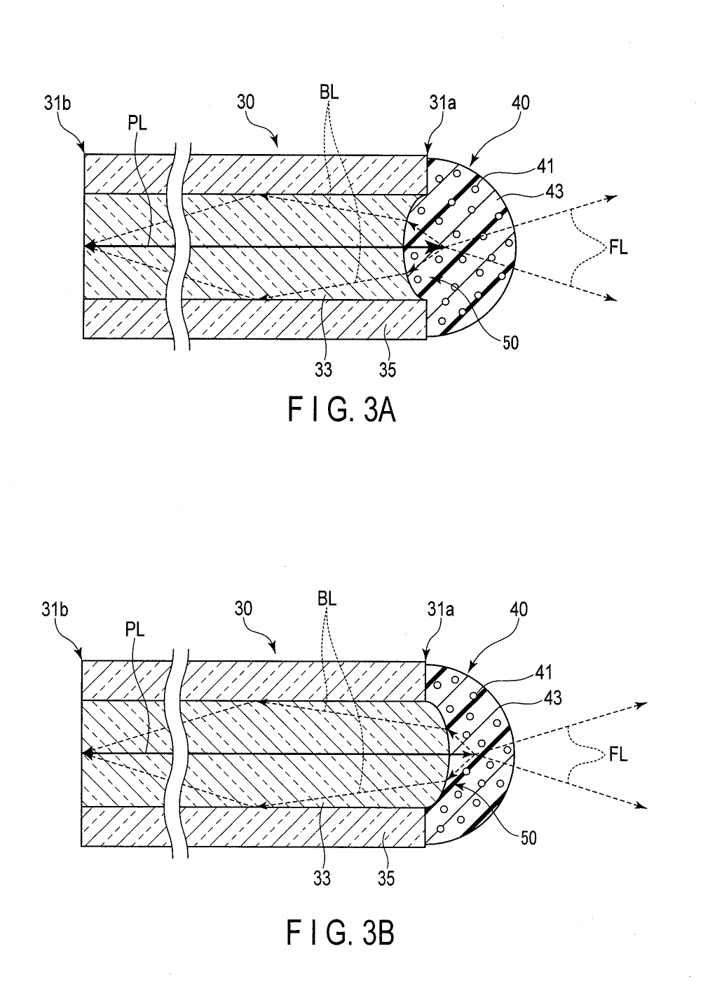

As illustrated in FIG. 3A, the light guide 30 includes a core 33 which guides the primary light PL and backward illumination light BL, and a cladding 35 which is provided on an outer periphery of the core 33 and has a refractive index that is lower than the refractive index of the core 33. In the light collection portion 50, the distal end surface of the core 33, which is included in the distal end surface 31a, is a concave surface. The refractive index of the core 33 is substantially equal to or lower than the refractive index of a contact part of the light converter 40, the contact part being in contact with the distal end surface of the core 33. The distal end surface of the cladding 35 may include a concave surface which is, for example, continuous with the core 33, or may be a planar surface.

Thereby, a lens effect occurs at the interface between the core 33 and the light converter 40, and the light distribution of the backward illumination light BL, which is incident on the core 33, can be narrowed. As a result, compared to the first embodiment, a greater amount of light can be made fall within the NA of the light guide 30, and the backward illumination light BL can efficiently be collected.

[Modification 2]

As illustrated in FIG. 3B, the light guide 30 includes a core 33 which guides the primary light PL and backward illumination light BL, and a cladding 35 which is provided on an outer periphery of the core 33 and has a refractive index that is lower than the refractive index of the core 33. In the light collection portion 50, the distal end surface of the core 33, which is included in the distal end surface 31a, is a convex surface. The refractive index of the core 33 is substantially equal to or higher than the refractive index of a contact part of the light converter 40, the contact part being in contact with the distal end surface of the core 33. The distal end surface of the cladding 35 may include a convex surface which is, for example, continuous with the core 33, or may be a planar surface.

Thereby, Modification 2 can obtain the same advantageous effects as Modification 1.

### Second Embodiment

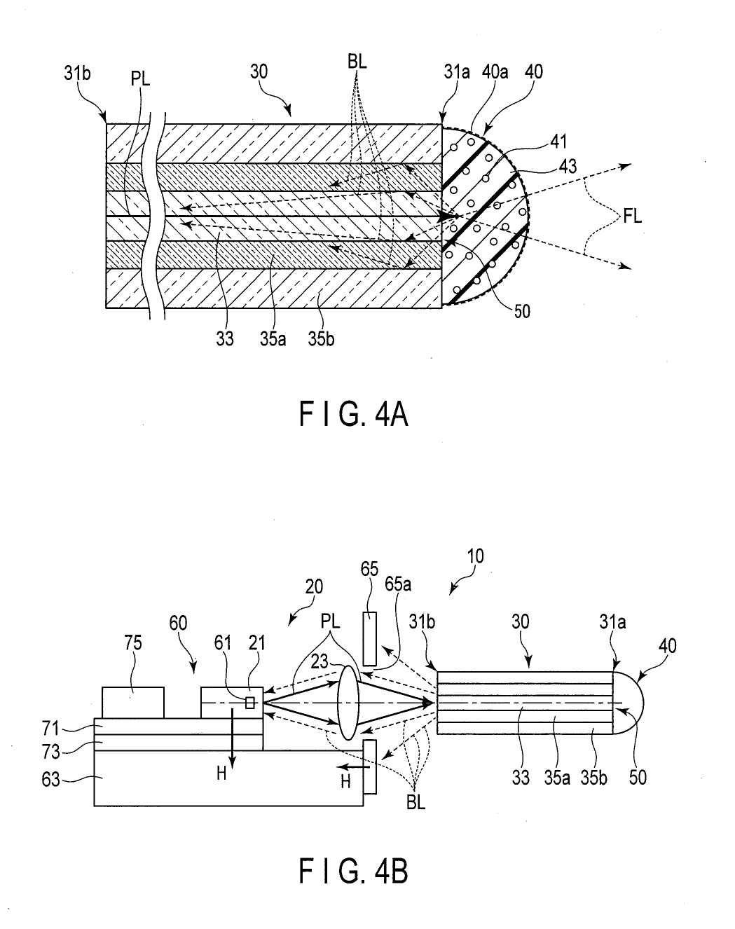

Hereinafter, referring to FIG. 4A and FIG. 4B, only the points different from the first embodiment will be described.

As illustrated in FIG. 4A, in the light guide 30, the optical fiber is a double-cladding fiber including a core 33, a first cladding 35a which is provided on an outer periphery of the core 33 and has a refractive index that is lower than the refractive index of the core 33, and a second cladding 35b which is provided on an outer periphery of the first cladding 35a and has a refractive index that is lower than the refractive index of the first cladding 35a. The light collector 50 includes a distal end surface of the core 33 and a distal end surface of the first cladding 35a in the distal end surface 31a, and includes the light converter 40.

As illustrated in FIG. 4A, when the light guide 30 guides primary light PL which is emitted from the light source 21, the core 33 guides the primary light PL. When the light guide 30 guides backward illumination light BL, the core 33 and first cladding 35a guide the backward illumination light BL.

When the optical fiber is the double-cladding fiber, backward illumination light BL with a high NA, which failed to be reflected at the interface between the core 33 and first cladding 35a, is exactly reflected at the interface between the first cladding 35a and second cladding 35b, and is exactly confined in the optical fiber. In addition, the backward illumination light BL is exactly guided to the heat exhauster 60. Thus, the heat generation of the distal end portion of the insertion module 121, in which the light converter 40 is provided, can be suppressed.

As illustrated in FIG. 4B, the heat exhauster 60 further includes an additional heat converter (additional heat conversion portion) 65 which is disposed on the outside of the optical path of the primary light PL. The additional heat converter 65 includes a hole (hole portion) 65a, through which the primary light PL can pass, and has a cylindrical shape. A part of the additional heat converter 65 is directly attached to the heat radiator 63, such that the hole 65a is disposed between the light focusing portion 23 and the proximal end surface 31b of the light guide 30 in the direction of travel of the primary light PL, and that the primary light PL passes through the hole 65a. The additional heat converter 65 is irradiated with the backward illumination light BL which is emitted from the core 33 and first cladding 35a. The additional heat converter 65 is formed of a member which has a high heat conductivity, and has a surface coated with a light absorbing film. The additional heat converter 65 is formed of, for example, aluminum or brass.

In the present embodiment, the light emission element, which is the heat converter 61, and the additional heat converter 65 are provided, and these components convert the backward illumination light BL to the heat H in a sharing manner. Thus, the heat generation of the distal end portion of the insertion module 121, in which the light converter 40 is provided, can be suppressed, the temperature rise of the light source 21 can be suppressed, and the light source 21 can stably be driven.

The large/small relationship of the NA will be described. As regards the NA of the primary light PL, the NA of the backward illumination light BL which is emitted from the core 33, and the NA of the backward illumination light BL which is emitted from the first cladding 35a, it is assumed that the NA of the primary light PL is lowest, the NA of the backward illumination light BL, which is emitted from the core 33, is second highest, and the NA of the backward illumination light BL, which is emitted from the first cladding 35a, is highest. In accordance with this, the size of the hole 65a is adjusted. Specifically, if the hole 65 has such a size as to pass most of the primary light PL, the backward illumination light BL, which is emitted from the core 33 and first cladding 35a, can irradiate the additional heat converter 65, and the backward illumination light BL can be converted to the heat H by the additional heat converter 65. Thereby, the heat generation of the distal end portion of the insertion module 121, in which the light converter 40 is provided, can be suppressed, the temperature rise of the light source 21 can be suppressed, and the light source 21 can stably be driven since the light source 21 is not irradiated with the backward illumination light BL.

In the meantime, as illustrated in FIG. 4A, a surface 40a of the light converter 40 may be formed to have asperities. In order to form asperities, the diffusion particles 41 may be exposed to the surface 40a of the light converter 40 by adjusting the density of the diffusion particles 41, or the surface of the enclosing member 43 may be formed to have asperities. Thereby, the reflection at an interface between the surface of the light converter 40 and external air can be reduced.

### Third Embodiment

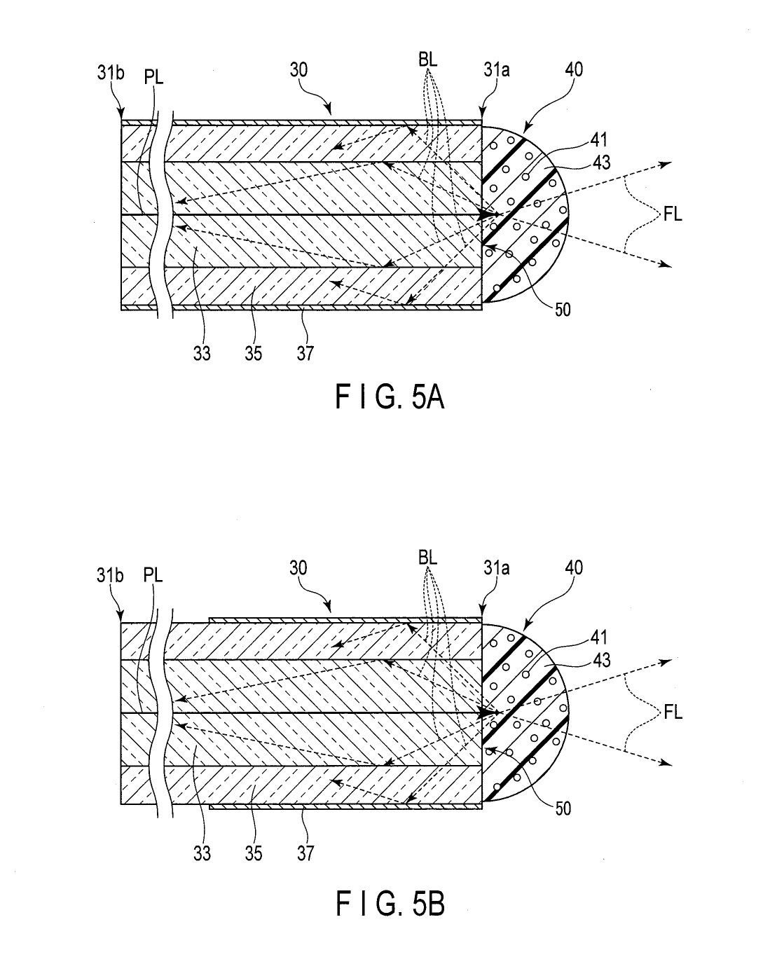

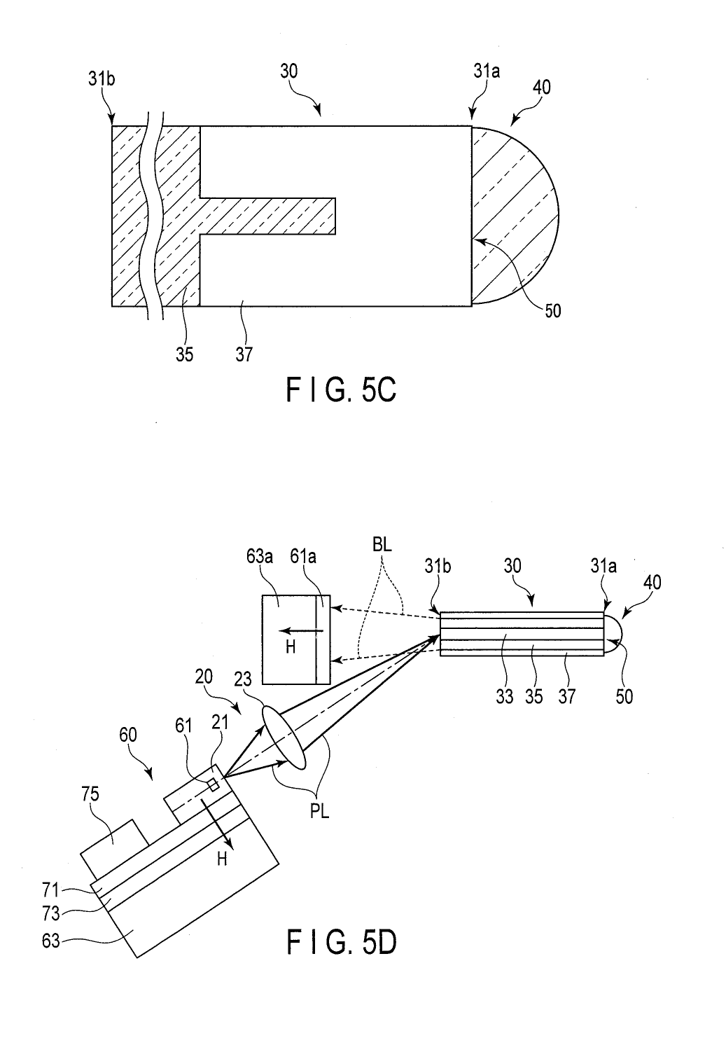

Hereinafter, referring to FIG. 5A, FIG. 5B, FIG. 5C and FIG. 5D, only the points different from the first and second embodiments will be described.

As illustrated in FIG. 5A, the optical fiber includes a core 33, a cladding 35 which is provided on an outer periphery of the core 33 and has a refractive index that is lower than the refractive index of the core 33, and a reflection film 37 which is provided on an outer periphery of the cladding 35 and is configured to reflect the backward illumination light BL, which is emitted from the cladding 35, toward the cladding 35. The light collector 50 includes a distal end surface of the core 33 and a distal end surface of the cladding 35 in the distal end surface 31a, and includes the light converter 40.

The reflection film 37 is formed of a member which has a high reflectance with respect to the wavelength of the backward illumination light BL. Such reflection film 37 is formed of, for example, gold, silver, aluminum, or nickel. The reflection film 37 is provided, for example, over the entire circumference of the cladding 35, and is continuous over the entire peripheral edge of the distal end surface 31a which is a part where the optical fiber is connected to the light converter 40. For example, in the axial direction of the optical fiber, the reflection film 37 is provided from the distal end surface 31a, which is the part where the optical fiber is connected to the light converter 40, to the proximal end surface 31b. In this manner, the reflection film 37 is provided on the entirety of the optical fiber. When this reflection film 37 is provided, backward illumination light BL with a high NA, which failed to be reflected at the interface between the core 33 and cladding 35, is exactly reflected by the reflection film 37, and is exactly confined in the optical fiber. In addition, the backward illumination light BL is exactly guided to the heat exhauster 60. Thus, the heat generation of the distal end portion of the insertion module 121, in which the light converter 40 is provided, can be suppressed.

In the meantime, as illustrated in FIG. 5B and FIG. 5C, the reflection film 37 may be provided on only a part of the optical fiber.

The reflection film 37 is provided, for example, over the entire circumference of the cladding 35, and is continuous over the entire peripheral edge of the distal end surface 31a which is a part where the optical fiber is connected to the light converter 40. In the axial direction of the optical fiber, the reflection film 37 is provided over only a predetermined length from the distal end surface 31a toward the proximal end surface 31b. In the vicinity of the light converter 40, the backward illumination light BL is confined in the optical fiber by the reflection film 37, and leaks out from the optical fiber at a location apart from the light converter 40. Thus, the backward illumination light BL can be converted to heat at a location apart from the light converter 40. In addition, the heat generation of the distal end portion of the insertion module 121, in which the light converter 40 is provided, can be suppressed.

As illustrated in FIG. 5C, the reflection film 37 is further provided only partly in the circumferential direction of the optical fiber, between the location apart by the above-described predetermined length and the proximal end surface 31b. The reflection film 37 does not reach the proximal end surface 31b, and is further provided over a predetermined length from the location apart by the above-described predetermined length. In this case, locations where the backward illumination light BL leaks from the optical fiber can be distributed, and local heat generation can be avoided. In this case, the reflection film 37 may be provided linearly along the axial direction of the optical fiber, or may be provided in a curved shape. This reflection film 37 may be provided up to the proximal end surface 31b.

As illustrated in FIG. 5D, a heat converter (heat conversion portion) 61a is disposed on an extension line of the optical axis of the light guide 30. The optical axis means, for example, the center axis of the backward illumination light BL which is emitted from the proximal end surface 31b. This heat converter 61a is additionally provided, separately from the light emission element of the light source 21, which is the heat converter 61. The heat converter 61a is thermally connected to a heat radiator (heat radiation portion) 63a. The heat radiator 63a radiates heat to the outside. This “outside” means, for example, an outside environment of the endoscope 120, or an atmosphere within the endoscope 120.

The light emission element of the light source 21, which is disposed in the light source module 20 and emits the primary light PL, is disposed in a position different from a position on the extension line of the optical axis. The light emission element is inclined to the optical axis, such that the primary light PL is incident on the light guide 30 within the NA of the optical fiber, with an inclination to the light guide 30.

Thus, the heat generation of the distal end portion of the insertion module 121, in which the light converter 40 is provided, can be suppressed, the temperature rise of the light source 21 can be suppressed, and the light source 21 can stably be driven.

[Others]

The double-cladding fiber of the second embodiment can be combined with the configurations of the first and third embodiments and the configurations of Modifications 1 and 2 of the first embodiment.

The additional heat converter 65 of the second embodiment can be combined with the configurations of the first and third embodiments and the configurations of Modifications 1 and 2 of the first embodiment.

The reflection film 37 of the third embodiment can be combined with the configurations of the first and second embodiments and the configurations of Modifications 1 and 2 of the first embodiment.

The configuration of the third embodiment, in which the light emission element of the light source 21 is disposed in a position different from a position on the extension line of the optical axis, can be combined with the configurations of the first and second embodiments and the configurations of Modifications 1 and 2 of the first embodiment.

### Fourth Embodiment

Referring to FIG. 6A and FIG. 6B, a description is given of an endoscope system 110 including the illumination apparatus 10 of the first embodiment. Incidentally, in the present embodiment, although the illumination apparatus 10 of the first embodiment is, by way of example, mounted in the endoscope system 110, the restriction to this is unnecessary. The illumination apparatus 10 of the other embodiments may be mounted. In the present embodiment, for the purpose of clearer illustration, the depiction of the heat exhauster 60, base plate 71, Peltier element 73 and temperature measuring sensor 75 is omitted.

[Endoscope System 110]

An endoscope system 110 as illustrated in FIG. 6A is installed, for example, in an examination room or an operating room. The endoscope system 110 includes an endoscope 120 which captures an image of, for example, an inside of a conduit (conduit portion) such as a lumen of a patient or the like, and an image processor (image processing apparatus) 130 which processes the image of the inside of the conduit, the image being captured by an imager (imaging unit (for example, CCD, CMOS), not shown) of the endoscope 120. The endoscope system 110 further includes a display (display portion) 140 which is connected to the image processor 130 and displays the image which was processed by the image processor 130, and a light source module 20 which emits primary light PL for illumination light L that is emitted from the endoscope 120. The display 140 has a monitor, for example.

The endoscope 120 as illustrated in FIG. 6A functions, for example, as an insertion apparatus which is inserted into the conduit. The endoscope 120 may be a forward-viewing endoscope 120 or a side-viewing endoscope 120.

The endoscope 120 of the present embodiment is described as being, for example, an endoscope 120 for medical use, but the restriction to this is unnecessary. The endoscope 120 may also be an endoscope 120 for industrial use, which is inserted in a conduit of an industrial product, such as a pipe, or an insertion instrument, such as a catheter, which includes only an illumination optical system.

As illustrated in FIG. 6A, the endoscope 120 includes an insertion module 121 which is hollow and elongated and is inserted into, for example, the conduit such as the lumen; and an operation portion 123 which is coupled to a proximal end portion of the insertion module 121 and operates the endoscope 120. The endoscope 120 includes a universal cord 125 which is connected to the operation portion 123 and is made to extend from a side surface of the operation portion 123.

As illustrated in FIG. 6A, the insertion module 121 includes a housing (housing portion) 121a which is provided on at least a part of the insertion module 121 and has flexibility. This housing 121a includes, for example, a flexible tube (flexible tube portion).

As illustrated in FIG. 6A, the operation portion 123 includes a housing (housing portion) 123a having desired rigidity.

As illustrated in FIG. 6A, the universal cord 125 includes a housing (housing portion) 125a which has flexibility and has desired rigidity. The universal cord 125 includes a connector (connection portion) 125b which is attachable/detachable to/from the image processor 130 and light source module 20. The connector 125b detachably connects the light source module 20 and endoscope 120 to each other, and detachably connects the endoscope 120 and image processor 130 to each other. The connector 125b is provided in order to enable data transmission/reception between the endoscope 120 and image processor 130.

The image processor 130 includes a housing (housing portion) 130a having desired rigidity.

Although not illustrated, the image processor 130 and light source module 20 are electrically connected to each other.

As illustrated in FIG. 6A, the light source module 20 includes a housing (housing portion) 20a having desired rigidity. The light source module 20 is a separate body from the endoscope 120, and is provided on an outside of the endoscope 120.

[Illumination Apparatus 10]

As illustrated in FIG. 6B, the endoscope system 110 further includes an illumination apparatus 10 which emits illumination light L toward the outside from the distal end portion of the insertion module 121.

As illustrated in FIG. 6A, the illumination apparatus 10 includes the above-described light source module 20; a light guide path 171 that is the above-described light guide 30, which is provided in the light source module 20 and in the endoscope 120 including the insertion module 121, is optically connected to the light source 21 of the light source module 20, and guides the primary light PL which is emitted from the light source 21; and the above-described light converter 40.

[Light Source 21V, 21B, 21G, 21R]

As illustrated in FIG. 6B, in the light source module 20, a plurality of light sources 21 can be provided. In the description below, the respective light sources 21 are referred to as light sources 21V, 21B, 21G and 21R. The light sources 21V, 21B, 21G and 21R are mounted on a control board (not shown) which forms a controller (control portion) 153 that controls the light sources 21V, 21B, 21G and 21R individually, and the controller 153 is electrically connected to a controller (control portion) 155. The controller 155 controls the entirety of the endoscope system 110 including the endoscope 120, display 140 and light source module 20. The controller 155 may be arranged in the image processor 130. The controller 153 and the controller 155 have, for example, a hardware circuitry including ASIC.

The light sources 21V, 21B, 21G and 21R emit primary lights PL having mutually optically different wavelengths. The light sources 21V, 21B, 21G and 21R emit, for example, the primary lights PL having high coherence, such as laser beams.

The light source 21V includes, for example, a laser diode which is a light emission element (heat converter 61) that emits a violet laser beam. A central wavelength of the laser beam is, for example, 405 nm.

The light source 21B includes, for example, a laser diode which is a light emission element (heat converter 61) that emits a blue laser beam. A central wavelength of the laser beam is, for example, 445 nm.

The light source 21G includes, for example, a laser diode which is a light emission element (heat converter 61) that emits a green laser beam. A central wavelength of the laser beam is, for example, 510 nm.

The light source 21R includes, for example, a laser diode which is a light emission element (heat converter 61) that emits a red laser beam. A central wavelength of the laser beam is, for example, 630 nm.

The light emission elements (heat converters 61) of the light sources 21V, 21B, 21G and 21R are disposed in the insides of housings (housing portions) 25V, 25B, 25G and 25R of the respective light sources 21V, 21B, 21G and 21R. In addition, light focusing portions 23 are disposed in the housings 25V, 25B, 25G and 25R.

Each of the light sources 21V, 21B, 21G and 21R is optically connected to a light coupler (light coupling portion) 157 (to be described later) via a single light guide (light guide member) 171a. The light guide 171a includes, for example, an optical fiber. Primary lights PL, which are emitted from the light emission elements of the light sources 21V, 21B, 21G and 21R, are focused on the single light guides 171a by the light focusing portions 23. Then, the primary lights PL are guided to the light coupler 157 by the light guides 171a. The light sources 21V, 21B, 21G and 21R, the controllers 153 and 155, and single light guides 171a are provided in the inside of the housing 20a.

For example, when white illumination is performed, the light source 21B, light source 21G and light source 21R are used. If four or more light sources 21 are provided, white-light observation using white light with high color rendering properties can be performed. When the light source 21V and light source 21G are used, special-light observation utilizing light absorption properties of hemoglobin can be performed. In the special-light observation, a blood vessel is displayed with emphasis. When a light source 21 which emits near-infrared light is used, observation utilizing near-infrared light can be performed. The light source 21 can be selected in accordance with the observation. In the present embodiment, visible light is used, but the restriction to this is unnecessary.

[Light Coupler 157]

As illustrated in FIG. 6B, the illumination apparatus 10 further includes the light coupler 157 which is provided in the inside of the housing 20a of the light source module 20, and couples the plurality of primary lights PL, which are emitted from the light sources 21V, 21B, 21G and 21R, into single light.

The light coupler 157 makes the primary lights PL, which are guided by the four light guides 171a, incident on a single light guide (light guide member) 171b. In this manner, in the present embodiment, the light coupler 157 includes four input ports and one output port. The number of input ports is equal to the number of light sources 21. The number of output ports is not particularly limited. At the input ports, the light guides 171a include fine optical fibers, and the light guides 171a are bundled. At the output port, the light guide 171b includes a thick optical fiber. The thick light guide 171b has a greater thickness than the bundled light guides 171a. The thick light guide 171b is fused on the bundled light guides 171a such that the thick light guide 171b is optically connected to the bundled light guides 171a. The light coupler 157 functions as a light combiner.

[Light Separator 159]

As illustrated in FIG. 6B, the illumination apparatus 10 further includes a light separator (light separating portion) 159 which is provided in the inside of the housing 20a of the light source module 20, and separates the primary light PL, which was coupled by the light coupler 157, into a plurality of primary lights PL.

The light separator 159 makes the primary light PL, which was guided by the single light guide 171b, incident on, for example, two light guides (light guide members) 171c. In this manner, in the present embodiment, the light separator 159 includes one input port and two output ports. The number of input ports of the light separator 159 is equal to the number of output ports of the light coupler 157. The number of output ports is not limited, if this number is plural. In other words, it should suffice if the number of light guides 171c is plural. The light separator 159 separates the primary light PL, for example, at a desired ratio. In this embodiment, the ratio is, for example, 50:50. It is not necessary that the ratio be equal between the respective output ports. The light separator 159 functions as a coupler.

In the structure of the light separator 159, the light guide 171b and one of the light guides 171c are one piece. In other words, the light guide 171b and one of the light guides 171c function as a member in common, for example, as an optical fiber in common. Another light guide 171c is fused to this one light guide, and the fused portion is further melted and drawn. Thereby, the primary light PL is transferred between the light guide 171b and the other light guide 171c.

In the present embodiment, the input port of the light separator 159 is optically connected to the output port of the light coupler 157. Thereby, the primary light PL, which is input to the light separator 159, is separated into the two light guides 171c at a ratio of, for example, 50:50.

Incidentally, although not illustrated, the light separator 159 may be provided in an inside of the housing 123a of the operation portion 123 of the endoscope 120. In this manner, it should suffice if the light separator 159 is provided in either the light source module 20 or the endoscope 120.

As illustrated in FIG. 6B, when the light separator 159 is provided in the light source module 20, the light guide 171b is provided in the inside of the housing 20a of the light source module 20, and the light guides 171c are provided in the inside of the housing 20a of the light source module 20 and in an inside of the endoscope 120. Although not illustrated, when the light separator 159 is provided in the endoscope 120, the light guide 171b is provided in the inside of the housing 20a of the light source module 20 and in the inside of the endoscope 120, and the light guides 171c are provided in the inside of the endoscope 120.

[Light Guide Path 171]

As illustrated in FIG. 6B, the light guide path 171 includes the above-described light guides 171a which are provided in the light source module 20. The light guides 171a are optically connected to the light sources 21 and the light coupler 157. The light guides 171a guide the primary lights PL from the light sources 21V, 21B, 21G and 21R to the light coupler 157.

The light guide path 171 further includes the light guide 171b which is provided in the light source module 20 when the light separator 159 is provided in the light source module 20 as illustrated in FIG. 6B, and which is provided in the light source module 20, connector 125b, universal cord 125 and operation portion 123 when the light separator 159 is provided, although not illustrated, in the operation portion 123. The light guide 171b guides the primary light PL from the light coupler 157 to the light separator 159.

The light guide path 171 further includes the light guides 171c which are provided in the light source module 20, connector 125b, universal cord 125, operation portion 123 and insertion module 121 when the light separator 159 is provided in the light source module 20 as illustrated in FIG. 6B, and which is provided in the operation module 123 and insertion module 121 when the light separator 159 is provided, although not illustrated, in the operation portion 123. The light guides 171c are optically connected to light converters 40. The light guides 171c guide the primary lights PL, which are emitted from the light source module 20, from the light separator 159 to the light converters 40. The light guides 171c may be directly connected to the light converters 40, or may be indirectly connected to the light converters 40 via a member (not shown, for example, lens).

As illustrated in FIG. 6B, the light guides 171c, which are provided in the insertion module 121, are provided in the inside of the housing 121a of the insertion module 121.

The light guides 171a, 171b and 171c include single optical fibers. In this embodiment, these single optical fibers are provided over the entirety of the light guide path 171, but the restriction to this is unnecessary. It should suffice if single optical fibers are provided on at least a part of the light guide path 171. If single optical fibers are provided on a part of the light guide path 171, a bundle fiber may be provided on the other part of the light guide path 171.

The single optical fibers functioning as the light guides 171a guide the primary lights PL which were emitted from the light sources 21.

In the light guides 171c, a plurality of single optical fibers are provided, and the optical fibers are single fibers of mutually different systems. In other words, these optical fibers are different members although these optical fibers have the same optical function of light guiding. Moreover, in other words, the light guides 171c include a plurality of single optical fibers of one kind, respectively. In this case, the light guides 171c function not as a bundle fiber, but as single optical fibers. The respective single optical fibers of the light guides 171a, 171b and 171c are single fibers of mutually different systems, and, in other words, these optical fibers are mutually different members although having the same optical function of light guiding.

As illustrated in FIG. 6B, when the light separator 159 is provided in the light source module 20, the light guides 171c, which are provided in the light source module 20, are different members from the light guides 171c which are provided on the connector 125b side.

Although not illustrated, when the light separator 159 is provided in the operation portion 123, the light guide 171b, which is provided in the light source module 20, is a different member from the light guide 171b which is provided on the connector 125b side.

The light guide 30 of the first embodiment functions as the light guides 171a, 171b and 171c.

Here, a brief description is given of a method in which the light guides 171c provided on the light source module 20 side, as illustrated in FIG. 6B, are optically connected to the light guides 171c provided on the connector 125b side.

As regards the light guides 171c provided in the light source module 20, the light guides 171c are inserted in a plug (plug unit) 191 which is provided in the light source module 20 and holds the light guides 171c.

The above-described content also applies to the light guides 171c provided on the connector 125b side. The plug unit 191 on the connector 125b side is provided in the connector 125b.

As illustrated in FIG. 6B, the housing 20a of the light source module 20 includes a light adapter 193 which is fixed to the housing 20a. The plug unit 191 on the light source module 20 side is attached in advance to the light adapter 193.

If the connector 125b is connected to the light source module 20, the plug unit 191 on the connector 125b side is inserted in the light adapter 193. Thereby, the light guides 171c on the light module side 20 side are optically connected to the light guides 171c on the connector 125b side. The plug unit 191 on the connector 125b side is attachable/detachable to/from the light adapter 193 of the light source module 20.

[Light Converter 40]

As illustrated in FIG. 6B, the light converters 40 are provided in the inside of the distal end portion of the insertion module 121. The light converters 40 are optically connected to the light guides 171c, and convert the primary lights PL, which are guided by the light guides 171c, to illumination light L. The light converters 40 emit the illumination light L to the outside of the endoscope 120, and irradiate the to-be-illuminated part with the illumination light L.

[Heat Exhauster 60]

Although illustration is omitted, in the present embodiment, the heat exhauster 60, base plate 71, Peltier element 73 and temperature measuring sensor 75 are provided in the inside of the housing 20a.

[Function]

Primary lights PL are emitted from the light emission elements of the light sources 21V, 21B, 21G and 21R, and are focused on the light guides 171a by the light focusing portions 23. The primary lights PL are guided to the light coupler 157 by the light guides 171a, and are coupled by the light coupler 157. The coupled primary light PL is guided to the light separator 159 by the light guide 171b, and is separated by the light separator 159. The separated primary lights PL are guided to the light converters 40 by the light guides 171c.

The light guide portion 40 diffuses the primary light PL, and the forward illumination light FL and backward illumination light BL are generated. The forward illumination light FL irradiates the to-be-illuminated part.

Like the first embodiment, the backward illumination lights BL are collected into the cores 33 of the light guides 171c by the light collectors 50 which are not shown in FIG. 6A and FIG. 6B. The backward illumination lights BL are guided to the light separator 159 by the light guides 171c, and are coupled by the light separator 159 which has also the function of the light coupler 157. The coupled backward illumination light BL is guided to the light coupler 157 by the light guide 171b. The backward illumination light BL is separated by the light coupler 157 which has also the function of the light separator 159, and the separated backward illumination lights BL are returned to the light sources 21V, 21B, 21G and 21R by the light guides 171a. In this manner, the backward illumination light BL is guided in the direction reverse to the direction of the travel of the primary light PL, reversely travels in the light guide path 171 in the direction reverse to the direction of the travel of the primary light PL, and returns to the light source 21V, 21B, 21G, 21R.

In the light sources 21V, 21B, 21G and 21R, the backward illumination lights BL are focused by the light focusing portions 23 on the respective light emission elements of the light sources 21V, 21B, 21G and 21R, which are the heat converters 61. Each light emission element, which is the heat converter 61, absorbs the backward illumination light BL, and converts the absorbed backward illumination light BL to heat H. The heat H is radiated to the outside by the heat radiator 63 via the base plate 71 and Peltier element 73, the depiction of which is omitted in FIG. 6A and FIG. 6B. This “outside” means, for example, an outside environment of the endoscope 120, or an atmosphere within the endoscope 120.

The heat converter 61 and heat radiator 63 convert the light to the heat H at a location apart from the light converter 40. Thus, in the present embodiment, the heat generation of the distal end portion of the insertion module 121 at a time of illumination, in which the light converter 40 is provided, can be suppressed to a minimum.

[Advantageous Effects]

In the present invention, even when the endoscope system 110 includes the illumination apparatus 10, the same advantageous effects as in the first embodiment can be obtained.

[Modification 1]

Referring to FIG. 7A and FIG. 7B, Modification 1 of the fourth embodiment will be described. Incidentally, in the present modification, for the purpose of clearer illustration, the depiction of the heat exhauster 60, base plate 71, Peltier element 73 and temperature measuring sensor 75 is omitted.

In the endoscope system 110 illustrated in FIG. 6A and FIG. 6B, the endoscope 120 is directly connected to various apparatuses via the universal cord 125 including the connector 125b.

However, in the present modification, as illustrated in FIG. 7A and FIG. 7B, the universal cord 125 is omitted, and the endoscope 120 is configured as a wireless type. In this case, the endoscope 120 is of such a wireless type that radio signals are transmitted/received between the operation portion 123 and image processor 130.

In addition, the endoscope 120 incorporates the illumination apparatus 10.

The illumination apparatus 10 of the present modification uses illumination light L of a narrow band. Thus, as illustrated in FIG. 7B, for example, light sources 21V and 21B are provided.

[Radio Unit in Illumination Apparatus 10]

As illustrated in FIG. 7B, the illumination apparatus 10 includes a radio (radio portion) 201 which is provided in the image processor 130 and outputs radio signals for controlling, for example, the light sources 21V and 21B and an imager (imaging unit (for example, CCD, CMOS)); and a controller (control portion) 203 which is electrically connected to the radio 201 and controls the endoscope system 110. The radio 201 and controller 203 are provided in the inside of the housing 130a having desired rigidity.

As illustrated in FIG. 7B, in the present modification, the light sources 21V and 21B are provided in the inside of the housing 123a of the operation portion 123.

As illustrated in FIG. 7B, the illumination apparatus 10 further includes a radio (radio portion) 211 which receives a radio signal that was output from the radio 201; and a controller (control portion) 213 which controls the light sources 21V and 21B, based on the radio signal received by the radio 211. The radio 211 and controller 213 are provided in the inside of the housing 123a of the operation portion 123. The light sources 21V and 21B are mounted on a control board (not shown) on which the controller 213 is formed.

As illustrated in FIG. 7B, the illumination apparatus 10 further includes a supplier (supply portion) 215 which supplies energy to the radio 211, controller 213 and light sources 21V and 21B. The supplier 215 is provided in the inside of the housing 123a of the operation portion 123. The supplier 215 includes, for example, a battery which supplies energy that is electric power. The supplier 215 also supplies energy to the respective members of the endoscope 120.

The above-described radio 201, controller 203, radio 211 and controller 213 function as a radio unit of the illumination apparatus 10 which is mounted in the wireless-type endoscope system 110. The controller 203 and the controller 213 have, for example, a hardware circuitry including ASIC.

The radio 201 may transmit a signal, which includes a driving condition of the light sources 21V and 21B, to the radio 211. Based on this driving condition, the controller 213 controls the light sources 21V and 21B.

The radio 211 may generate a video signal, based on an imaging signal of a to-be-illuminated part which was imaged by the imager (not shown), may convert the video signal to a radio signal, and may transmit the radio signal to the radio 201. The controller 203 converts the radio signal to a video signal, and executes image processing on the video signal. The display 140 displays the video signal as a video image.

The radio 211 may transmit residual amount information, which indicates a residual amount of energy in the supplier 215, to the radio 201. In addition, the display 140 may display this residual amount information.

In this manner, various pieces of information are transmitted/received between the radios 201 and 211.

[Light Coupler/Separator 217]

As illustrated in FIG. 7B, the light sources 21V and 21B are provided in the inside of the housing 123a of the operation portion 123. Thus, in consideration of the space in the housing 123a, the illumination apparatus 10 includes a light coupler/separator (light coupling/separating portion) 217 which is provided in the inside of the housing 123a of the operation portion 123 and has the function of the light coupler 157 and the function of the light separator 159 in the first embodiment. The light coupler/separator 217 functions as a light combiner and a coupler.

As illustrated in FIG. 7B, the light coupler/separator 217 is optically connected to a light guide (light guide member) 171a which is optically connected to the light source 21V, and also optically connected to a light guide (light guide member) 171a which is optically connected to the light source 21B. The light coupler/separator 217 is further optically connected to light guides (light guide members) 171c which are optically connected to the light converters 40. In this manner, the light coupler/separator 217 includes two input ports and two output ports. The number of input ports of the light coupler/separator 217 is equal to the number of light sources 21. The number of output ports is not particularly limited, if the number is plural. In other words, it should suffice if the number of light guides 171c is plural.

The light coupler/separator 217 couples the primary light PL which was emitted from the light source 21V and guided by the light guide 171a, and the primary light PL which was emitted from the light source 21B and guided by the light guide 171a.

The light coupler/separator 217 separates the coupled primary light PL into a plurality of primary lights PL. The light coupler/separator 217 separates the primary light PL, for example, at a desired ratio. In this modification, the ratio is, for example, 50:50. It is not necessary that the ratio be equal between the respective output ports.

[Heat Exhauster 60]

Although illustration is omitted, in the present modification, the heat exhauster 60, base plate 71, Peltier element 73 and temperature measuring sensor 75 are provided in the inside of the housing 123a.

In the present modification, the illumination apparatus 10 is included in the wireless-type endoscope 120, but the restriction to this is unnecessary. The illumination apparatus 10 may be included in the endoscope 120 illustrated in the fourth embodiment.

[Advantageous Effects]

In the present modification, even when the endoscope 120 incorporates the illumination apparatus 10, the same advantageous effects as in the first and second embodiments can be obtained.

[Modification 2]

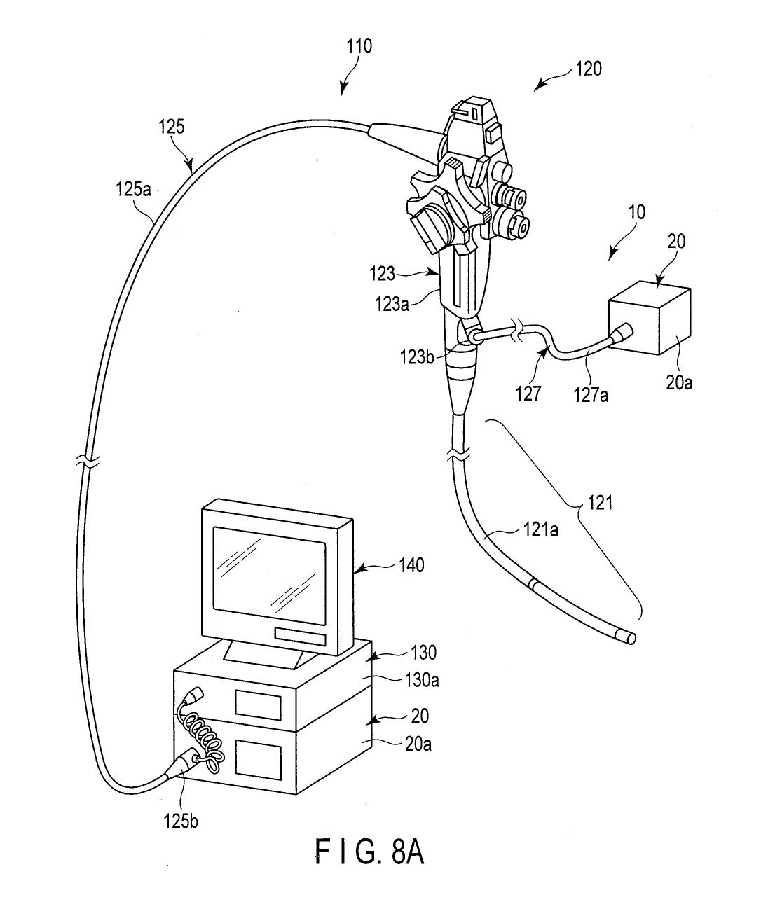

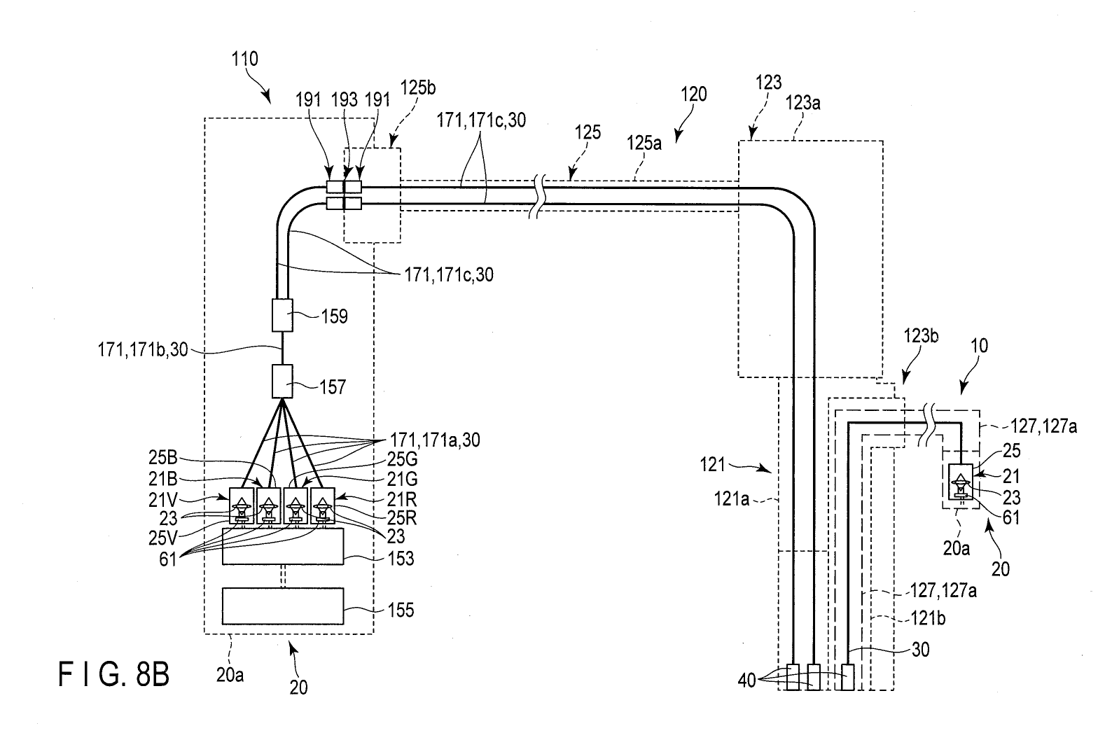

Referring to FIG. 8A and FIG. 8B, Modification 2 of the fourth embodiment will be described. Incidentally, in the present modification, for the purpose of clearer illustration, the depiction of the heat exhauster 60, base plate 71, Peltier element 73 and temperature measuring sensor 75 is omitted. A light source 21 includes a housing (housing portion) 25 in which a light emission element (heat converter 61) and a light focusing portion 23 are included.

As illustrated in FIG. 8A and FIG. 8B, the illumination apparatus 10 may be inserted into a treatment instrument insertion channel 121b from a treatment instrument insertion port (treatment instrument insertion portion) 123b. In this case, the endoscope 120 is a separate body from the illumination apparatus 10. The illumination apparatus 10 is insertable/removable into/from the endoscope 120.

A light guide 30 is inserted through a housing (housing portion) 127a of an auxiliary universal cord 127. The housing 127a has flexibility and has desired rigidity. The light guide 30 is inserted through the treatment instrument insertion channel 121b via the housing 127a, such that the light converter 40 is disposed in the distal end portion of the insertion module 121.

The auxiliary universal cord 127 is fixed to the housing 20a. The light converter 40 is fixed to the distal end portion of the housing 127a.

[Advantageous Effects]

In the present modification, even when the illumination apparatus 10 is inserted through the treatment instrument insertion channel 121b, the same advantageous effects as in the first embodiment can be obtained.

In the state in which the endoscope system 110 and endoscope 120 include the illumination apparatus 10 in advance, an illumination apparatus 10 is additionally provided. Thereby, a greater amount of illumination light L can be irradiated on a to-be-observed object. In short, in the present modification, the illumination apparatus 10 can also function as an auxiliary illumination apparatus 10.

Incidentally, in the present modification, it is not necessary that the endoscope system 110 and endoscope 120 include the illumination apparatus 10 in advance, as illustrated in FIG. 7A and FIG. 7B and in FIG. 8A and FIG. 8B. In this case, the configuration of the endoscope system 110 and endoscope 120 can be simplified.

Although illustration is omitted, in the present modification, the heat exhauster 60, base plate 71, Peltier element 73 and temperature measuring sensor 75 are provided in the inside of the housing 20a.

The endoscope 120 of the present modification may be of a wireless type as illustrated in Modification 1.

The present invention is not limited directly to the above-described embodiments. At the stage of practicing the invention, the structural elements may be modified and embodied without departing from the spirit of the invention. Various inventions may be made by suitably combining a plurality of structural elements disclosed in the embodiments.

What is claimed is:

1. An illumination apparatus comprising:
a light source module configured to emit primary light;
a light guide configured to guide the primary light emitted from the light source module;
a light converter disposed on a distal end surface of the light guide, the light converter being configured to emit illumination light, which is generated by converting optical characteristics of the primary light that is guided by the light guide, in a forward direction which is on the light converter side of the distal end surface, and in a backward direction which is on the light guide side of the distal end surface;
a light collector configured to collect backward illumination light, which is the illumination light emitted backward from the light converter, into the light guide, such that the backward illumination light is guided backward by the light guide; and
a heat exhauster configured to convert the backward illumination light, which is guided by the light guide, to heat, and to exhaust the heat.

2. The illumination apparatus according to claim 1, wherein the light collector includes the distal end surface and the light converter.

3. The illumination apparatus according to claim 2, wherein the light guide includes a core configured to guide the primary light and the backward illumination light, and a cladding which is provided on an outer periphery of the core and has a refractive index that is lower than a refractive index of the core, and
a distal end surface of the core, which is included in the distal end surface, is a planar surface in the light collector.

4. The illumination apparatus according to claim 3, wherein the refractive index of the core is substantially equal to or higher than a refractive index of a contact part of the light converter, the contact part being in contact with the distal end surface of the core.

5. The illumination apparatus according to claim 2, wherein the light guide includes a core configured to guide the primary light and the backward illumination light, and a cladding which is provided on an outer periphery of the core and has a refractive index that is lower than a refractive index of the core, and
a distal end surface of the core, which is included in the distal end surface, is a concave surface in the light collector.

6. The illumination apparatus according to claim 5, wherein the refractive index of the core is substantially equal to or lower than a refractive index of a contact part of the light converter, the contact part being in contact with the distal end surface of the core.

7. The illumination apparatus according to claim 2, wherein the light guide includes a core configured to guide the primary light and the backward illumination light, and a cladding which is provided on an outer periphery of the core and has a refractive index that is lower than a refractive index of the core, and
a distal end surface of the core, which is included in the distal end surface, is a convex surface.

8. The illumination apparatus according to claim 7, wherein the refractive index of the core is substantially equal to or higher than a refractive index of a contact part of the light converter, the contact part being in contact with the distal end surface of the core.

9. The illumination apparatus according to claim 1, wherein the light guide includes an optical fiber.

10. The illumination apparatus according to claim 9, wherein the optical fiber is a multi-mode optical fiber configured to guide a plurality of modes of the primary light and the backward illumination light.

11. The illumination apparatus according to claim 10, wherein the optical fiber has such an NA that the optical fiber receives 20% or more of the backward illumination light.

12. The illumination apparatus according to claim 10, wherein the optical fiber includes a core configured to guide the primary light and the backward illumination light, and a cladding which is provided on an outer periphery of the core and has a refractive index that is lower than a refractive index of the core, and
a diameter of the cladding is not greater than 1.1 times a diameter of the core.

13. The illumination apparatus according to claim 9, wherein the optical fiber is a double-cladding fiber including a core, a first cladding which is provided on an outer periphery of the core and has a refractive index that is lower than a refractive index of the core, and a second cladding which is provided on an outer periphery of the first cladding and has a refractive index that is lower than the refractive index of the first cladding.

14. The illumination apparatus according to claim 13, wherein the core is configured to guide the primary light when the light guide guides the primary light which is emitted from the light source module, and
the core and the first cladding are configured to guide the backward illumination light when the light guide guides the backward illumination light.

15. The illumination apparatus according to claim 9, wherein the optical fiber includes a core, a cladding which is provided on an outer periphery of the core and has a refractive index that is lower than a refractive index of the core, and a reflection film which is provided on an outer periphery of the cladding and is configured to reflect the backward illumination light, which is emitted from the cladding, toward the cladding.

16. The illumination apparatus according to claim 15, wherein the reflection film is provided over an entire circumference of the cladding, and is continuous over an entire peripheral edge of the distal end surface of the light guide, the distal end surface being a part where the optical fiber is connected to the light converter.

17. The illumination apparatus according to claim 16, wherein, in an axial direction of the optical fiber, the reflection film is provided from the distal end surface of the light guide to a proximal end surface of the light guide, or the reflection film is provided over only a predetermined length from the distal end surface of the light guide toward the proximal end surface of the light guide.

18. The illumination apparatus according to claim 17, wherein the reflection film is further provided only partly in a circumferential direction of the optical fiber, between a location apart by the predetermined length and the proximal end surface of the light guide.

19. The illumination apparatus according to claim 1, wherein the heat exhauster includes a heat converter configured to absorb the backward illumination light and converts the absorbed backward illumination light to heat, and a heat radiator configured to radiate the heat.

20. The illumination apparatus according to claim 19, wherein the heat converter is a light emission element which is included in the light source module and which is configured to emit the primary light.

21. The illumination apparatus according to claim 20, wherein the heat exhauster further includes an additional heat converter disposed on an outside of an optical path of the primary light.

22. The illumination apparatus according to claim 19, wherein the heat converter is further disposed on an extension line of an optical axis of the light guide, and
the light emission element, which is disposed in the light source module and configured to emit the primary light, is disposed in a position different from a position on the extension line of the optical axis.

23. The illumination apparatus according to claim 1, wherein the light converter functions as a light distribution converter configured to convert a light distribution of the primary light.

24. The illumination apparatus according to claim 23, wherein the light converter includes one or more diffusion particles which diffuse the primary light, and an enclosing member configured to enclose the diffusion particles together in a state in which the diffusion particles are dispersed, and
the light converter is formed in a dome shape.

25. The illumination apparatus according to claim 24, wherein the enclosing member is formed of a member which transmits the primary light.

26. The illumination apparatus according to claim 24, wherein a refractive index of the diffusion particle is different from a refractive index of the enclosing member.

27. The illumination apparatus according to claim 24, wherein a diameter of the diffusion particle is about 1/10 or more of a wavelength of the primary light.

28. The illumination apparatus according to claim 24, wherein in a cross section in an optical axis direction of the light guide, a central angle of an outer arc of the light converter having the dome shape is 180 degrees or less.

29. The illumination apparatus according to claim 24, wherein a surface of the light converter is formed to have asperities.

30. The illumination apparatus according to claim 24, wherein the heat exhauster is configured to convert 5% or more of the backward illumination light, which is emitted backward by the light converter, to the heat.

31. An endoscope comprising the illumination apparatus according to claim 1.

32. An endoscope configured such that the illumination apparatus is inserted into a treatment instrument insertion channel from a treatment instrument insertion port portion, separately from the illumination apparatus according to claim 1.

33. An endoscope system comprising the illumination apparatus according to claim 1.
